(19) United States
(12) Patent Application Publication
(10) Pub. No.: US 2023/0054079 A1
(43) Pub. Date: Feb. 23, 2023

(54) PULSE FIELD ABLATION FOR THE TREATMENT OF MIGRAINES

(71) Applicant: Natraj Lakshmi, SHOREWOOD, MN (US)

(72) Inventor: Natraj Lakshmi, SHOREWOOD, MN (US)

(21) Appl. No.: 17/892,509

(22) Filed: Aug. 22, 2022

Related U.S. Application Data
(60) Provisional application No. 63/236,202, filed on Aug. 23, 2021.

(51) Int. Cl. A61B 18/14; A61B 18/12

(52) U.S. Cl. CPC A61B 18/1492; A61B 18/1206; A61B 2017/0019

(57) ABSTRACT

Described herein is a method of treating migraines using PFA ablation technology which includes advancing a pulse field ablation delivery member into a nasal cavity (both unilateral and bilateral) of a patient with the pulse field ablation member in a first collapsed configuration and contacting a surface of a nasal cavity tissue with the pulse field ablation delivery member without penetrating or piercing the nasal cavity tissue surface. The treatment further includes reconfiguring the pulse field ablation delivery member from the first collapsed configuration to an expanded configuration after introducing the pulse field ablation member into the desired position within the nasal cavity, and ablating a target treatment site with the pulse field ablation delivery member in order to treat or prevent at least one of the group of medical conditions, wherein the target treatment site includes at least one nasal nerve tissue or nasal blood vessel with or without contact.

### CLAIM OF PRIORITY

This application claims priority to and the benefit of U.S. Provisional Application with Ser. No. 63/236,202, filed on Aug. 23, 2021, with the same title, which is hereby incorporated by reference in its entirety.

### FIELD OF THE INVENTION

The inventive concept relates generally to a method and a system for treating headaches and migraines.

### BACKGROUND

Migraines are a neurological disorder with symptoms of pain primarily in the head, mostly around the eyes and templates of an afflicted patient. There are different classifications and intensities of migraines. In the US alone, there are over 13% of adults that suffer from migraines or severe headaches (32 Million patients in US) and over 3.4 Million visit to healthcare providers, including visits to emergency rooms in the debilitating cases.

Currently there are various ways to treat migraines, spanning from pharmacology, BOTOX injections to external stimulating devices (using modalities such as TENS, . . . etc.). However, there is a great unmet need as there are a sizable subset of migraine patients that are refractory to above treatments. Cryotherapy has been considered for ablating Sphenopalatine Ganglion tissue, but the treatment time is longer and equipment needed is more expensive, making it a less attractive commercial option for medical professionals.

Therefore, there currently exists a need in the market for a system and a method promptly provides relief to patients in a cost effective and noninvasive manner, particularly where patients cannot tolerate continued or any pharmaceutical treatment.

### SUMMARY OF THE INVENTION

It would be advantageous to have a treatment method and a system for providing noninvasive treatment to patients of migraine symptoms, particularly on an outpatient basis and with no pharmaceutical component that may interact with other medications or impair the motor skills of the patient. Various studies indicate the neurological connections between Sphenopalatine Ganglion (hereinafter SPG) and Migraines. Studies have also included how impacting the SPG positively influences migraines. A novel method of treatment is to stimulate the Sphenopalatine Ganglion for treating Headaches and Migraine is via Pulsed Field Ablation (hereinafter (PFA). In the last decade, there has been substantial advancement in the field of pulsed field ablation for treating cardiac arrhythmias by ablating cardiac tissues. Pulsed Field Ablation is a recent method for interventional treatment, first used in cardiac arrhythmia treatment. It leverages the localized application of short (micro or nano second) high voltage electric fields to induce irreversible electroporation, a prolonged state or pore-induced permeability that triggers non-necrotic deaths of cells and targeted tissue.

In one example embodiment of the treatment method is to leverage Pulsed Field Ablation technology by similarly introducing a probe/catheter into the nasal cavity and thereafter ablate one or more targeted locations, such as but not limited to the middle meatus, superior meatus and inferior meatus thereby impacting or stimulating the bundle of nerves at the SPG.

In one example embodiment of the system of treating migraines, there is provided a pulse field ablation generator operatively coupled to a handheld device having a proximal end and a distal end. The proximal end of the handheld device is connected to the generator while the distal end containing a treatment probe with an electrode tip is configured to be introduced into the nasal cavity. Using an endoscope to accurately visualize the meatus region in the nasal cavity, the surgeon could identify the exact location to ablate and leverage the Pulse Field Ablation technology to ablate the targeted region.

In a related embodiment, a method of treating migraines and other upper respiratory debilitating conditions using PFA ablation technology is provided which includes advancing a pulse field ablation delivery member into a nasal cavity (both unilateral and bilateral) of a patient with the pulse field ablation member in a first collapsed configuration and contacting a surface of a nasal cavity tissue with the pulse field ablation delivery member without penetrating or piercing the nasal cavity tissue surface. The treatment further includes reconfiguring the pulse field ablation delivery member from the first collapsed configuration to an expanded configuration after introducing the pulse field ablation member into the desired position (or target treatment site) within the nasal cavity, and stimulating or ablating a target treatment site with the pulse field ablation delivery member via a train of pulses having a predetermined time duration and amplitude level or voltage level, wherein the target treatment site includes at least one nasal nerve tissue or nasal blood vessel.

Although a nerve block is a known procedure to stop pain transmission through anesthesia to the nerve. In an SPG block, an anesthetic agent is administered to the collection of nerves in the ganglion. The least invasive way to access the SPG is through the nose as this SPG block serves to support stimulation/ablation of the SPG nerves and region.

The inventive concept now will be described more fully hereinafter with reference to the accompanying drawings, which are intended to be read in conjunction with both this summary, the detailed description and any preferred and/or particular embodiments specifically discussed or otherwise disclosed. This inventive concept may, however, be embodied in many different forms and should not be construed as limited to the embodiments set forth herein; rather, these embodiments are provided by way of illustration only and so that this disclosure will be thorough, complete and will fully convey the full scope of the inventive concept to those skilled in the art.

### BRIEF DESCRIPTION OF THE DRAWINGS

FIGS. 1A and 1B illustrate an unassembled and an assembled, respectively, pulse field ablation (PFA) delivery member according to the teachings herein.

FIG. 2 illustrates a system that includes a pulse field generator and a control module operatively coupled to a PFA treatment delivery probe for treating migraines and acute headaches in a patient.

FIG. 3 is a graphic representation or illustration of a human head highlighting the various nerves around and including the sphenopalatine ganglion (SPG) and the nasal mucosa potentially subject to PFA ablation according to the teachings herein.

FIG. 4 illustrates an anatomical illustration of a human head identifying various nerves around and including the sphenopalatine ganglion (SPG) subject to PFA ablation according to the teachings herein.

FIG. 5 illustrates a partial anatomical illustration of a human head identifying various nerves around and including the sphenopalatine ganglion (SPG) subject to PFA ablation according to the teachings herein.

FIG. 6 illustrates a flowchart of a method of treating migraines and migraine-like systems in a human or mammal using PFA ablation according to the teachings herein.

### DETAILED DESCRIPTION OF THE INVENTION

Following are more detailed descriptions of various related concepts related to, and embodiments of, methods and apparatus according to the present disclosure. It should be appreciated that various aspects of the subject matter introduced above and discussed in greater detail below may be implemented in any of numerous, ways, as the, subject matter is not limited to any particular manner of implementation. Examples of specific implementations and applications are provided primarily for illustrative purposes.

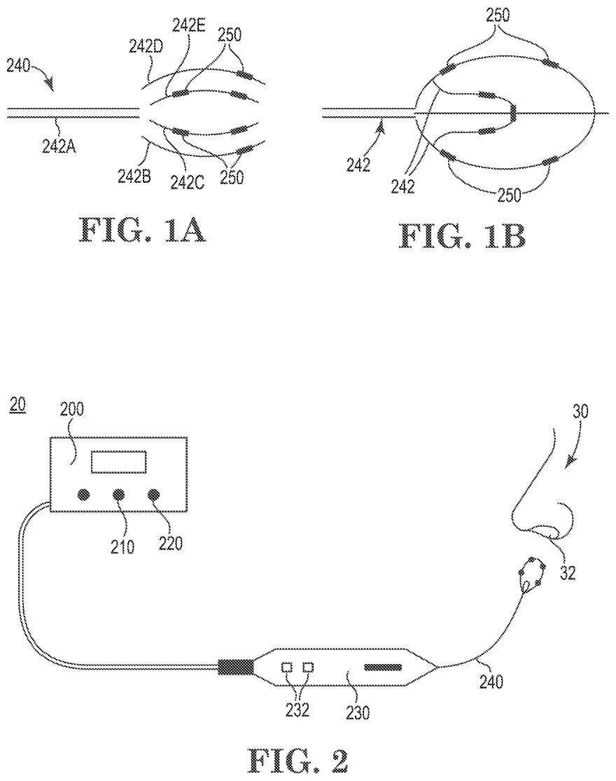

Referring now to FIGS. 1A-3, there is described a system 20 that is primarily for, but not necessarily limited to tissue ablation, which is accomplished through selective and rapid application of electric pulse waveforms primarily in the form of trains of individual pulses. In this example embodiment, system 20 for ablation tissues includes at least the following components housed in a housing 200, a pulse field generator 210 and a control module 220, and a handheld treatment delivery device 230 with an adaptive probe 240. In this non-limiting example embodiment, adaptive probe 240 includes a conductor 242 and a plurality of electrodes 250, located at a tip or distal end of probe 240, which deliver electrical pulses for ablation of tissue in a patient 30. The probe or catheter 240 is adaptive to the nasal cavity anatomy. The generator 210 may provide electrical pulses to the medical device or treatment probe 240 to provide ablation to the nerves accessed through the nasal cavity.

Referring briefly to FIGS. 1A and 1B, there is illustrated an unassembled probe 240 have various separate conductors 242A-242E, with various electrode elements 250 that are electrically connected to form conductor 242 as shown in FIG. 1B. Multiple electrodes 250 may deliver, in a defined or treatment area of the nasal cavity, biphasic or monophasic pulses having a pre-programmed pattern and waveform for therapeutic purposes to treat headaches and migraines. In this example embodiment, handheld device 230 includes control buttons 232 and is electrically coupled to housing 200 with provides power through pulse generator 210 and system intelligence (such as operating software and associated applications) through control module 220. Handheld device 230 supports at its distal end 236 probe 240 for maneuvering the probe and conductor 240 into nasal cavity 32 of patient 30. In a related embodiment, system 20 provides for a medical device or handheld device 230 proximate the nasal/neural tissue to be treated with the medical device including a distal electrode array having a multiple electrodes and with each electrode having a polarity that is different than each adjacent electrode, and where the pulse train of energy is delivered from the distal electrode array, such as electrodes 250. In yet another related embodiment, prove 240 and its conductors are configured to be disposable after each patient use to promote patient health and safety. In yet another embodiment, a disposable sheath is configured to cover probe 240 and the conductors to allow for treatment of the patient and patient hygiene and safety.

In this example embodiment, device 230 and probe 240 are designed to provide a pulse train of energy (similar to square waves, that have a certain time duration and an amplitude representative of a voltage level) having a voltage between 5 V and 5000V with a predetermined or defined range of frequencies. In a related embodiment, the inter-pulse delay could be 100 to 900 microseconds, using mono or biphasic pulses. Further, the pulse train of energy can be delivered to a plurality of nasal/neural treatment sites using mono or biphasic pulses, with or without contact.

In other related embodiments, the pulse train of energy is delivered in any one of the modes in a group consisting of: unipolar, bipolar mode, mono or biphasic mode, with multiple voltages. The generator 210 may include an electrical current or pulse generator having a plurality of output channels, with each channel coupled to an individual electrode or multiple electrodes.

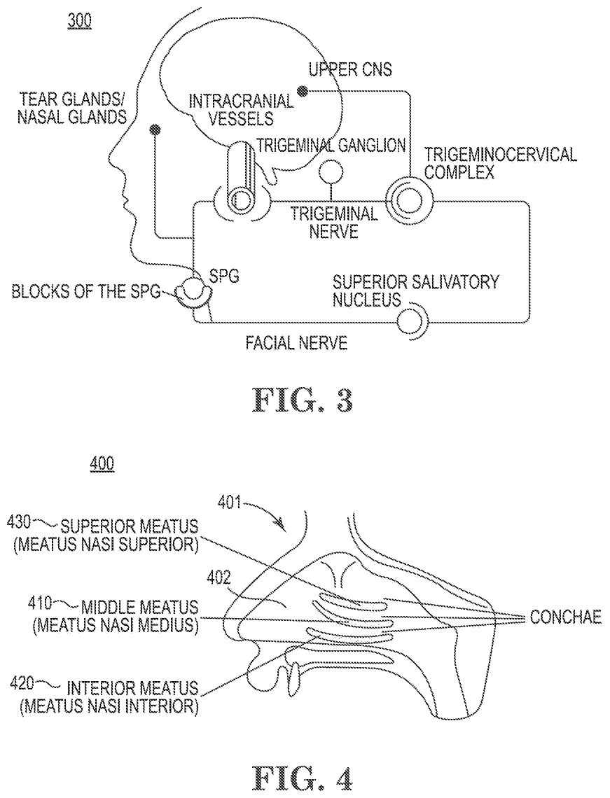

FIG. 3 is a diagram 300 illustrating the distance between the sphenopalatine ganglion (SPG) and nasal mucosa and demonstrating the involvement of the sphenopalatine ganglion in the physiopathology of trigeminal autonomic headaches. The afferent part of this loop is mediated by the trigeminal nerve, which sends nociceptive signals from the dural blood vessels to the trigeminocervical complex. This information projects to higher brain structures, resulting in cephalic pain. The efferent part of this loop conveys mostly through the superior salivatory nucleus, exiting the brain stem via the facial nerve and reaching the sphenopalatine ganglion through the greater petrosal nerve. Postganglionic fibers exit the sphenopalatine nerve towards the dural vessels, closing the loop. Blocking the SPG should reduce the afferent input of signals towards the trigeminal system and reduce the activation of the trigeminocervical complex.

Referring to FIG. 4 there is illustrated a partial anatomical illustration 400 of a human head 401 identifying various nerves around and including the sphenopalatine ganglion (SPG) which is subject to PFA ablation. In particular, the objective is to stimulate or ablate the Sphenopalatine Ganglion (SPG) through intranasal approach (nasal cavity 402) by ablating the middle meatus 410, inferior meatus 420, and posterior or superior meatus 430 deep within nasal cavity 402. Traditionally, the topical intranasal administration of local anesthesia in order to attempt to block the SPG had met with only moderate success. By pulsing with electric waveforms specifically taught herein, we expect superior outcomes.

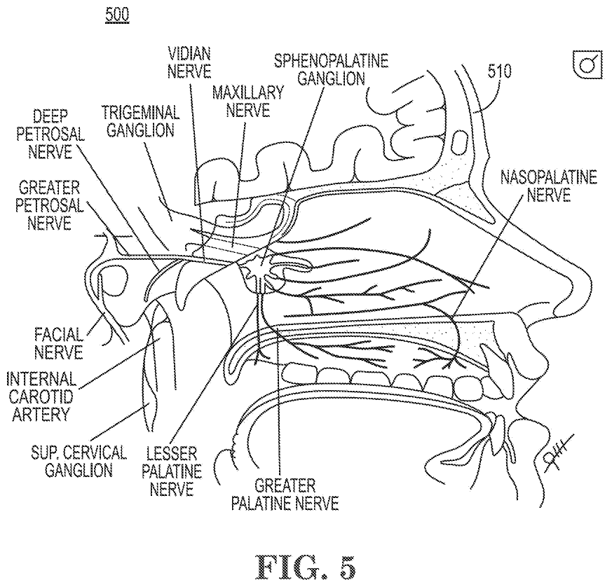

Referring now to FIG. 5 there is illustrated an anatomical illustration 500 of a human head 510 identifying various nerves around and including the sphenopalatine ganglion (SPG) that is subject to PFA ablation according to the teachings herein. The sphenopalatine ganglion (SPG) is a collection of nerve cells that is closely associated with the trigeminal nerve, which is the main nerve involved in headache disorders. It contains autonomic nerves and sensory nerves. Autonomic nerves are specialized nerves that control organ functions, including gut and bladder movements, beating of the heart, sweating, salivation, tearing and other secretions. In the SPG, these autonomic nerves supply the lacrimal glands (which produces tears) and the inner lining of the nose and sinuses (which produces nasal discharge or congestion). The SPG is located just behind the bony structures of the nose.

The SPG has connections to the brainstem (where cluster and migraine attacks may be generated) and to the meninges (coverings of the brain) by the trigeminal nerve. Inflammation and opening of the blood vessels around the meninges occur, which activate pain receptors that send pain impulses through the trigeminal nerve, eventually to the sensory area of the brain, and are perceived as pain. In migraine and cluster headache, nerves carrying these pain signals pass through the SPG, with some making connections to the autonomic nerves. This explains why in a cluster headache, and sometimes in a migraine, we see autonomic features including tearing of the eyes and nasal congestion or discharge. We call this the trigeminal autonomic reflex.

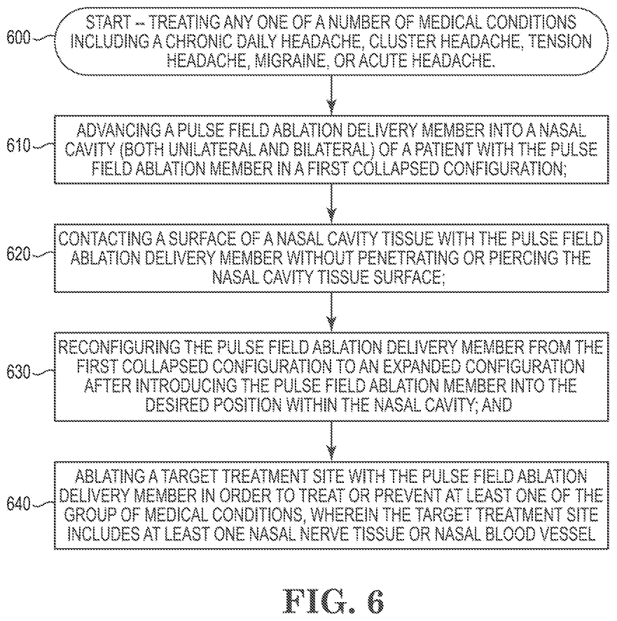

Referring now to FIG. 6, there is illustrated a flowchart of a method 600 of treating migraines and migraine-like symptoms in a human or mammal using PFA ablation according to the teachings herein. In particular, method 600 includes treating or preventing in a patient any one of a number of medical conditions in a group consisting of: a chronic daily headache, cluster headache, tension headache, migraine, or acute headache. Method 600 includes the step 610 of advancing a pulse field ablation delivery member into a nasal cavity (both unilateral and bilateral) of a patient with the pulse field ablation (PFA) member 240 in a first collapsed configuration and step 620 of contacting a surface of a nasal cavity tissue with the pulse field ablation delivery member 240 without penetrating or piercing the nasal cavity tissue surface.

Method 600 further includes step 630 of reconfiguring the pulse field ablation delivery member 240 from the first collapsed configuration to an expanded configuration after introducing the pulse field ablation member into a target treatment site or a desired position within the nasal cavity. Method 600 then includes step 640 of ablating the target treatment site with the pulse field ablation delivery member 240 via a train of pulses having a predetermined time duration and amplitude level or voltage level, wherein the target treatment site includes at least one nasal nerve tissue or nasal blood vessel. In particular, the train of pulses includes individual pulses with a time duration from 100-900 microseconds and a voltage level of 5V to 5000V, which can be monophasic or biphasic.

The following patents and publications are incorporated by reference in their entireties: U.S. Pat. Nos. 10,709,891; 10,617,467; 10,531,914; 9,999,465; US 2019/0254735; and PCT/US2019/042877; and literature including a neuroimaging study Crespi et al. The Journal of Headache and Pain (2018) 19:14 https://doi.org/10.1186/s10194-018-0843-5.

While the inventive concept has been described above in terms of specific embodiments, it is to be understood that the inventive concept is not limited to these disclosed embodiments. Upon reading the teachings of this disclosure many modifications and other embodiments of the inventive concept will come to mind of those skilled in the art to which this inventive concept pertains, and which are intended to be and are covered by both this disclosure and the appended claims. It is indeed intended that the scope of the inventive concept should be determined by proper interpretation and construction of the appended claims and their legal equivalents, as understood by those of skill in the art relying upon the disclosure in this specification and the attached drawings.

1. A method for treating or preventing in a patient any one of a number of medical conditions in a group consisting of: a chronic daily headache, cluster headache, tension headache, migraine, or acute headache, the method comprising:
advancing a pulse field ablation delivery member into a nasal cavity (both unilateral and bilateral) of a patient with the pulse field ablation member in a first collapsed configuration;
contacting a surface of a nasal cavity tissue with the pulse field ablation delivery member without penetrating or piercing the nasal cavity tissue surface;
reconfiguring the pulse field ablation (PFA) delivery member from the first collapsed configuration to an expanded configuration after locating the pulse field ablation member proximate a target treatment site within the nasal cavity; and
ablating the target treatment site with the pulse field ablation delivery member to ablate tissue in the nasal cavity via a train of pulses having a predetermined time duration and amplitude level or voltage level, wherein the target treatment site includes at least one nasal nerve tissue or nasal blood vessel.

2. The method of claim 1, wherein advancing the pulse field ablation delivery member into the nasal cavity includes further advancing the pulse field ablation delivery member into at least one of an inferior meatus, a middle meatus, or a superior meatus.

3. The method of claim 1 wherein the nasal blood vessel is a sphenopalatine artery.

4. The method of claim 1 wherein the nasal nerve tissue is a sphenopalatine ganglion (SPG), and wherein the sphenopalatine ganglion is ablated or pulsed through a perpendicular plate of a palatine bone within a superior meatus between a middle turbinate and an anterior face of a sphenoid sinus.

5. The method of any one of claim 1, wherein the nasal nerve tissue is any one of a group consisting of:
a posterior nasal nerve or an accessory posterior nasal nerve;
a nerve branch originating from the sphenopalatine ganglion;
an autonomic nerve branch; and
a sensory nerve fiber.

6. The method of any one of claim 1, wherein the nasal nerve tissue is a parasympathetic nerve fiber.

7. The method of claim 6, wherein the parasympathetic nerve fiber is a postsynaptic parasympathetic nerve fibers emerging from a sphenopalatine ganglion.

8. The method of claim 1, wherein advancing the pulse field ablation delivery member into the nasal cavity includes further advancing the pulse field ablation delivery member to a treatment site in the nasal cavity responsive to PFA ablation to treat for Rhinitis.

9. The method of claim 1, wherein advancing the pulse field ablation delivery member into the nasal cavity includes further advancing the pulse field ablation delivery member to a treatment site in the nasal cavity responsive to PFA ablation to treat a nasal obstruction.

10. A system for treating a patient having a medical condition related to an upper respiratory location, the medical condition being at least one of a group consisting of a chronic daily headache, cluster headache, tension headache, migraine, or acute headache, the system comprising:
a pulse field ablation (PFA) delivery member having a distal end and a proximal end, the distal end of the pulse field ablation delivery member adapted to have a probe tip with a plurality of electrodes for insertion into a nasal cavity of a patient; and
a pulse generator and control module adapted to be operatively coupled to the proximal end of the pulse field ablation delivery member, the pulse generator and control module adapted to energize the probe tip with the plurality of electrodes to ablate tissue in the nasal cavity via a train of pulses having a predetermined time duration and amplitude level or voltage level.

11. The system of claim 10, wherein the probe tip is adapted to stimulate various areas proximate to the SPG nerve tissue.

12. The system of claim 10, wherein the train of pulses includes individual pulses with a time duration from 100-900 microseconds and a voltage level of 5V to 5000V.

13. The system of claim 10, wherein the probe tip is adapted to ablate various areas proximate the SPG nerve tissue.

14. The system of claim 12 wherein the train of pulses are either mono or biphasic.

15. The method of claim 4 wherein the PFA delivery member includes a probe tip adapted to stimulate various areas proximate to the SPG nerve tissue.

16. The method of claim 15, wherein the probe tip provides a train of pulses that includes individual pulses with a time duration from 100-900 microseconds and a voltage level of 5V to 5000V.

17. The method of claim 15, wherein the probe tip is adapted to ablate various areas proximate the SPG nerve tissue.

18. The method of claim 16 wherein the train of pulses are either mono or biphasic.
